(19) United States
(12) Patent Application Publication
(10) Pub. No.: US 2020/0155722 A1
(43) Pub. Date: May 21, 2020

(54) Dermal Filler Composition

(71) Applicant: Matthew Kihara, Dublin, CA (US)

(72) Inventor: Matthew Kihara, Dublin, CA (US)

(73) Assignee: matthew kihara, Dublin, CA (US)

(21) Appl. No.: 16/356,661

(22) Filed: Apr. 18, 2019

Related U.S. Application Data
(63) Continuation of application No. 62/768,756, filed on Nov. 16, 2018.

(51) Int. Cl. A61L 27/26

(52) U.S. Cl. CPC A61L 27/26; A61L 2400/06; A61L 2430/34

(57) ABSTRACT

A permanent dermal filler composition may include a vinyl-containing polydimethylsiloxane (PDMS); a silicon hydride containing PDMS; and a platinum catalyst. The ratio of the hydride part of the polymer to the vinyl part of the polymer may be from about 0.2:1.0 to about 5.0:1.0.

### BACKGROUND OF THE INVENTION

The embodiments described herein relate generally to chemical compositions, and more particularly, to a dermal filler composition.

Current technology for dermal fillers are largely temporary and require several ongoing treatments to maintain the patient's desired look. Methods for permanent fillers use potentially harmful microspheres or unbound leaching molecules.

Therefore, what is needed is a dermal filler composition that allows for a permanent biocompatible fill without the need for plastic spheres or migratable fluids.

Some embodiments of the present disclosure include a permanent dermal filler composition. The dermal filler composition may include a vinyl-containing polydimethylsiloxane (PDMS); a silicon hydride containing PDMS; and a platinum catalyst. The ratio of the hydride part of the polymer to the vinyl part of the polymer may be from about 0.2:1.0 to about 5.0:1.0.

### DETAILED DESCRIPTION OF THE INVENTION

In the following detailed description of the invention, numerous details, examples, and embodiments of the invention are described. However, it will be clear and apparent to one skilled in the art that the invention is not limited to the embodiments set forth and that the invention can be adapted for any of several applications.

The composition of the present disclosure may be used as a permanent dermal filler and may comprise the following elements. This list of possible constituent elements is intended to be exemplary only, and it is not intended that this list be used to limit the composition of the present application to just these elements. Persons having ordinary skill in the art relevant to the present disclosure may understand there to be equivalent elements that may be substituted within the present disclosure without changing the essential function or operation of the composition.

The various elements of the composition of the present invention may be related in the following exemplary fashion. It is not intended to limit the scope or nature of the relationships between the various elements and the following examples are presented as illustrative examples only.

By way of example, some embodiments of the invention comprise a permanent dermal filler composition using a low durometer in-vivo curing cross-liked silicone as a backbone.

Hyaluronic acid (HA), collagen, or similar known non-permanent fillers may also be added to the uncured silicone and delivered together, allowing the silicone network to allow passage of biomaterials. By using a filler composition based on an in-vivo crosslinking silicone network, patients may have a permanent dermal filler solution that is both biocompatible and gives a natural flesh-like feel.

In particular, the permanent dermal filler composition may comprise a vinylcontaining polydimethylsiloxane (PDMS), a silicon hydride containing PDMS, and a platinum catalyst. In some embodiments, the ratio of the hydride part of the polymer to the vinyl part of the polymer may be about 0.2:1.0 to 5.0:1.0. A particular embodiment's vinyl to hydride ratio may be about 0.8:1. The composition may further comprise optional additives or co-dermal fillers, such as HA or collagen, to allow for a network that may allow the passage of biomaterials. Through a polyaddition reaction catalyzed by a platinum containing catalyst, the vinyl containing PDMS may crosslink with the silicon hydride containing PDMS to create a crosslinked silicone network. This reaction may be inhibited by freezing, allowing the composition to be delivered as a single-component dermal filler that, when heated to body temperature, will safely crosslink in-vivo. Thus, the ingredients may be blended, sterilized, packaged, and stored at a low temperature until it is time to inject the dermal filler into a patient. Optionally, common fugitive or competitive polyaddition silicone inhibitors may be used to tune the curing time at room or body temperature. In another embodiment, the platinum and silicon hydride containing PDMS may be separated to allow for a two-component injectable dermal filler that would not require the need for low temperature. However, this embodiment would add the complexity of mixing and injecting at the time of use in a safe, sterilized, and controlled manner.

By adding HA or collagen to the composition, the network formed would allow the passage of biomaterials.

While the composition of the present disclosure is described broadly as being a dermal filler composition, it may also be used as a cavity filler or soft tissue aesthetic, such as for breast implants.

The above-described embodiments of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claim.

### BRIEF DESCRIPTION OF THE DRAWING

The drawing shows the matrix possible with silicone itself or when combined with a co-filler.

What is claimed is:

1. A permanent dermal filler composition comprises:
a vinyl-containing polydimethylsiloxane (PDMS);
b silicon hydride containing PDMS; and
c platinum catalyst.

2. The permanent dermal filler composition of claim 1, wherein a ratio of the hydride part of the polymer to the vinyl part of the polymer is from about 0.2:1.0 to about 5.0:1.0.
